(19) United States
(12) Patent Application Publication
(10) Pub. No.: US 2009/0048293 A1
(43) Pub. Date: Feb. 19, 2009

(54) METHODS FOR TREATING BACILLUS INFECTION

(76) Inventors: Serguei Popov, Bristow, VA (US); Taissia Popova, Bristow, VA (US); Svetlana Nazarenko, Manassas, VA (US); Charles Bailey, Cross Junction, VA (US); Chris Bradburne, Bristow, VA (US); Bryan Millis, Bristow, VA (US); Vikas Chandhoke, Fairfax, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/913,518

(22) PCT Filed: May 5, 2006
(86) PCT No.: PCT/US2006/017421

§ 371 Date: Apr. 7, 2008

Related U.S. Application Data
(60) Provisional application No. 60/677,814, filed on May 5, 2005.

(51) Int. Cl. A61K 31/47; C12Q 1/02; C12N 9/50; A61P 31/00; C12Q 1/37; G01N 33/53

(52) U.S. Cl. 514/312; 435/29; 435/7.1; 435/219; 435/23; 604/5.02

(57) ABSTRACT

The present invention provides compositions and methods for detecting, treating, and preventing microbial infection, especially infection caused by Bacillus anthracis (“anthrax”).

### CROSS REFERENCE

This application claims the benefit of U.S. Provisional Application No. 60/677,814, filed on 5 May 2005, the entirety of which is herein incorporated by reference.

### BACKGROUND

The genus Bacillus is a diverse group of gram-positive bacteria that are characterized by their ability to form spores. Bacillis anthracis is the most well-known member of this genus since it is the causative agent of anthrax. Other members of the group also are associated with disease pathology, such as B. cereus. Moreover Bacillus and the infection they cause are similar to other gram-positive bacteria such as Staphylococcus aureus and Listeria monocytogenes, and can be treated in some of the same ways.

For instance, anthrax infection (i.e., infection by B. anthracis) occurs in three main forms: cutaneous, inhalation, and gastrointestinal. Cutaneous anthrax infection can occur when a bacterium enters the epithelium through a cut or abrasion on the skin. Infection begins as a raised itchy bump that resembles a small insect bite. Within 1-2 days, the bump develops into a vesicle and then a painless ulcer, usually about 1-3 cm in diameter, with a characteristic black necrotic area in its center. Lymph glands in the adjacent area may swell. About 20% of untreated cases of cutaneous anthrax will result in death. The initial symptoms of inhalational anthrax may resemble a common cold with sore throat, mild fever, muscle aches, and malaise. After several days, the symptoms may progress to severe breathing problems and shock. Inhalation anthrax is usually fatal. Gastrointestinal anthrax can occur when contaminated meat or other products comprising the bacterium are consumed. Initially, infected subjects will exhibit acute inflammation of the intestinal tract accompanied by nausea, loss of appetite, vomiting, and fever. These progress rapidly into abdominal pain, vomiting of blood, and severe diarrhea. Gastrointestinal anthrax results in death in 25% to 60% of cases. Anthrax pathology is described in more detail in Inglesby et al. JAAM 287:2236-2252, 2002.

Inhalation anthrax is a severe, often fatal disease characterized by systemic spread of the challenge agent, Bacillus anthracis, which is capable of causing severe damage to host tissues and organs. Multiple hemorrhagic lesions in the mediastinum, mediastinal lymph nodes, bronchi, lungs, heart, spleen, liver, intestines, kidneys, adrenal glands, and/or central nervous system are typically found upon postmortem examination of patients who succumbed to inhalation anthrax. The most dramatic and potentially life-threatening changes are observed in the vascular system with a diffuse vasculitis extending from moderate sized arteries and veins down to the capillary level. The vasculitis is often associated with vessel destruction, especially of the smallest vessels, and is typically accompanied by massive necrosis in some tissues.

It is widely believed that anthrax lethal toxin (LeTx) secreted by proliferating bacteria is a major cause of death in man and in several other susceptible animal species. However, the pathology of intoxication in experimental animals is drastically different from that found during the natural infectious process. Recent extensive analyses in mice and rats challenged with a highly purified lethal toxin confirmed earlier observations that toxin activity caused no gross pathology and almost solely manifested in hypoxic liver failure. These results have suggested that other factors are involved in the disease pathology.

Anthrax infection in humans can be a pernicious, quick, and often fatal disease. Because of this, together with its relative simplicity as an organism and its availability as robust dispersible infectious spores, anthrax has been among the few organisms of primary interest to biowarfare programs worldwide. The lethal effects of anthrax in humans have been amply demonstrated by the deaths caused by the accidental release of weaponized anthrax in the former Soviet Union. The stealth of weaponized anthrax also has been dramatically illustrated in the United States, more recently by the still unsolved murders of several postal workers exposed to anthrax sent through the mail.

Difficulties of aggregation, dispersion, and control of the released organism have largely rendered anthrax unattractive to conventional military strategists, quite apart from nearly universal treaties against its use. Such obstacles are unlikely to deter terrorists, however, and anthrax is among the organisms of most concern to anti-terrorist organizations.

Methods to detect anthrax and anthrax infection, defenses against anthrax infection, and treatments of persons infected with anthrax consequently, are a very high priority for both military and anti-terrorist organizations as well as public health organizations in the United States and abroad. The interests of those organizations include prophylactic measures (such as vaccination) as well as curative treatments. While there are currently available preventative measures and a number of effective treatments, none of them are entirely satisfactory due to, for instance, suboptimal or uncertain effectiveness, severe (occasionally fatal) side effects, dependence for effectiveness on early detection and immediate treatment, and/or only partial efficacy.

In addition to its human health hazards, anthrax infects a wide variety of other animals, including, among domesticated animals, a variety of economically important livestock animals. While infections of non-humans does not pose the same hazards and concerns as those discussed above regarding direct human infection, anthrax infection of animals poses both a secondary risk of human infection and a direct risk to livestock that is important to our food supply. The threat to and the effects of anthrax infection on animals likely exceeds the threat to humans, were it not for the possibility of a biowarfare attack using anthrax, and almost certainly exceeds considerably the actual harm to humans. Prevention of infection and limiting the spread of infection is a prime concern for animal anthrax, especially those of livestock animals, since infected animals generally must be sacrificed. Effective vaccines have been developed for several livestock species, but the cost apparently is too high for widespread prophylactic use. Early detection of exposure and infection thus is an important aspect of minimizing the destructive effects of anthrax infection of livestock animals.

Anthrax is merely illustrative of the diseases caused by Bacillus sp. and other gram negative bacteria. Collectively, these organisms cause a variety of diseases and engender thereby considerable suffering and economic damage. Among such organisms that are not of the Bacillus genus, are a variety of other gram negative organisms, such as, for instance, Staphylococcus sp. and other organisms described in greater detail below.

As for the detection, prevention, amelioration, and/or cure of human anthrax infections, the available reagents and methods for detecting, preventing, ameliorating, and/or curing anthrax infection in humans similarly applies to those for animals.

There is, therefore, a need for improved tools and methods for detecting, ameliorating, curing, and otherwise treating or dealing with such Bacillus-associated diseases, such as anthrax, and for dealing with disorders and diseases caused by other gram negative organisms similar in pertinent nature to Bacillus in this regard.

### BRIEF DESCRIPTION OF THE FIGURES

FIG. 1. Syndecan-1 release upon treatment of NMuMG cell with B. anthracis pathogenic factors and related proteins. The results of dot-blot were used for densitometry and plotted in arbitrary units.

FIG. 2. Dot-blot (left panel) and graphical representation (right panel) of syndecan-1 release in the blood of mice challenged with 30 LD50 of Sterne strain spores intraperitoneally. Data represent 2 mice at the day of challenge, 3 mice at each of days 1 and 2, and 2 mice at day 3.

FIG. 3. TLR2 response in HEK 293 cells upon treatment with B. anthracis pathogenic factors, culture supernatant (B.a. sup) diluted 8-fold, Cln A, AnlB, and AnlO. Treatment was carried out in the presence of 10% FCS for 24 h. Controls include bacterial (LB) and HEK cell media. Luminescence is shown in arbitrary units.

FIG. 4. TLR2 response in HEK 293 cells upon treatment with B. anthracis culture supernatant (B.a. sup) diluted 8-fold before and after heat inactivation (HI). Treatment was carried out in the presence of 0.5% FCS for 24 h. Controls include bacterial and HEK cell media. Luminescence is shown in arbitrary units.

FIG. 5. TLR2 response in HEK 293 cells in the presence of 10% FCS after 24 h induced by B. anthracis hemolysins indirectly through conditioned medias, which were obtained after incubation of NMuMG cells with 10 μg/ml of a particular hemolysin in presence of 10% FCS for 4 h. Controls include bacterial and HEK cell media. Luminescence is shown in arbitrary units.

FIG. 6. TLR4 response in HEK 293 cells upon treatment with B. anthracis culture supernatant (B.a. sup) diluted 8-fold in presence (open bars) and in absence (filled bars) of 25 μg/ml polymyxin. Treatment was carried out in the presence of 10% FCS for 24 h. Controls include bacterial and HEK cell media, and 1 μg/ml LPS. Luminescence is shown in arbitrary units.

FIG. 7. Inhibitors of protein tyrosine kinase (piceatannol, tyrphostin), heparinase (suramin), and metalloprotease (o-phenanthroline) reduce LeTx-induced syndecan-1 shedding. Y axis indicates integrated intensity of syndecan-1 signals (arbitrary units).

FIG. 8. Inhibitors of protein tyrosine kinase (piceatannol, tyrphostin), heparinase (suramin), metalloprotease (o-phenanthroline), and matrix metalloproteases (Galardin) reduce AnlO-induced syndecan-1 shedding. Y axis indicate integrated intensity of syndecan-1 signals (arbitrary units).

FIG. 9. Purification and identification of Npr599 and InhA. Two proteases were purified from a culture of B. anthracis delta Ames through ammonium sulfate saturation, DEAE-cellulose, and sephacryl S-200 column chromatography.
(A) Reduced SDS-PAGE gel of proteins in each purification step. M, prestained molecular markers (from top to bottom, 250, 148, 98, 64, 50, 36, 22, and 16 kDa); lane 1, culture supernatant; lane 2, ammonium sulfate saturation; lane 3, DEAE cellulose of P1; lane 4, DEAE-cellulose of P2; lane 5, sephacryl S-200 of P1; and lane 6, sephacryl S-200 of P2.
(B) Summary of the purification.
(C) The N-terminal amino acid sequences of the purified proteases.

FIG. 10. Potential substrates for Npr599 and InhA. Biologically important substrates were digested with 0.2 μg of Npr599 (P1), InhA (P2), and without protease (No) in each reaction for 4 hrs at 37° C. Boiled samples were separated by SDS-PAGE (10%, 14%, or 4-20%) stained with Coomassie blue. A. Digestion of extracellular matrix proteins (ECMs) was analyzed by SDS-14% PAGE for fibrinectin (FN, lanes 2-4) and laminin (LN, lanes 5-7), and SDS-10% PAGE for collagen type I (Col-I, lanes 9-11) and collagen type IV (Cil-IV, lanes 12-14). B. Digestion of endogenous serum protease inhibitors was analyzed by SDS-10% PAGE for α₂-macroglobulin (α₂-MG, lanes 25-27), α₁-proteinase inhibitor (PI, lanes 18-19), and SDS-4-20% PAGE for α₂-antiplasmin (α₂-AP, lanes 21-23). C. Digestion of immune response proteins was analyzed by SDS-4-20% PAGE for IgG (lanes 25-27), IgM (lanes 28-30), and SDS-10% PAGE for IgA (lanes 31-33), and interferon-γ (IFN-γ, lanes 34-36). D. digestion of blood coagulation or tissue damage related response proteins was analyzed by SDS-10% PAGE for fibrinogen (Fbg, lanes 38-40) and plasminogen (Plg, lanes 41-43). Lanes 1, 8, 24, and 37 represent molecular markers.

FIG. 11. Acceleration of urokinase-dependent plasminogen (Plg) activation by InhA. A. Npr599 (P1) and InhA (P2) are not a bacterial plasminogen activator. Human plasminogen (8.3 μg) was incubated at 37° C. with 2 μg of the protease or streptokinase (SK). The 20-fold diluted resulting reactions were added to 100 μM Val-Leu-Lys-pNA in the presence of fibrin and the release of pNA was monitored during the incubation. B. Urokinase-type plasminogen activator (uPA)-catalyzed plasminogen activation is accelerated by InhA. The reaction was achieved by adding 200 U/ml uPA, 0.1 U/ml plasminogen, 100 μM Val-Leu-Lys-pNA and with 2, 5, and 10 μg/ml of the purified proteases to the reaction solutions (100 μl). The release of pNA from the chromogenic substrate was monitored at 405 nm for first 10 min.

FIG. 12. Enhancement of syndecan-1 shedding by Npr599 and InhA. Confluent NMuMG cells in 96-well plates were incubated with (A) various concentrations (62.5, 250, and 500 ng/ml) of Npr599 and InhA for 4 h, or (B) 250 ng/ml protease for 1, 4, and 8 h at 37° C. Shed syndecan-1 ectodomain levels were measured by the dot-blot analysis as described in the Examples. Error bars represent S. D. determined from triplicate measurements.

FIG. 13. Effect of inhibitors on syndecan-1 ectodomain shedding from NMuMG cells enhanced by Npr599 and InhA. NMuMG cells in 1% FCS medium were preincubated with the indicated concentrations of inhibitors for 1 h, and then exposed to shedding inducers (250 ng/ml of either Npr599 or InhA) for 24 h. Data are expressed relative to shedding observed without inhibitors in cells either treated or untreated with Npr599 and InhA. Dotted and two-point chain lines represent control syndecan-1 ectodomain shedding by Npr599 and InhA in the absence of inhibitors, respectively. SB and PD represent SB202190 and PD98059, respectively. Error bars represent S. D. determined from triplicate measurements. Confidence intervals correspond to P=0.05.

FIG. 14. Direct cleavage of N-terminus of recombinant syndecan-1 by Npr599 and InhA. A. Recombinant syndecan-1 core protein tagged with GST (800 ng) was incubated without (lane 1) or with 100 ng of Npr599 (lane 2) and InhA (lane 3) for 4 h at 37° C., and analyzed on SDS-4-20% PAGE. After electrophoresis, gel was immunoblotted with antibody against GST. B. The immunoblot was incubated with antibody against N-terminus of syndecan-1 epitope. Lanes 1-3 are the same as legends of A. GST-SDC1 and GST-SDC (N-term) represent GST-fused syndecan-1 and N-terminal fragment of GST-SDC1, respectively. C Coomassie blue stained SDS-PAGE gel of GST-SDC1 after incubation without any protease (lane 1), Npr599 (200 ng, lane 2), InhA (200 ng, lane 3), LF (200 ng, lane 4), and LF (1 μg, lane 5).

FIG. 15. Western immunoblotting of syndecan-1 ectodomains shed by B. anthracis culture supernatant and purified proteases Npr599 and InhA. Syndecan-1 ectodomains from the conditioned media of unstimulated NMuMG cells (lane 1) and from NMuMG cells stimulated with 250 ng/ml of Npr599 (lane 2) and InhA (lane 3), 10% (v/v) B. anthracis Δ Ames culture supernatant in LB (lane 4) and in LB with 0.5% glucose (lane 5), or 1 μM PMA (lane 6) were separated by 4-20% SDS-PAGE gel electrophoresis. The shed syndecan-1 was transferred on a cationic immobilon (NY⁺) nylon membrane and immunoblotted with the 281-2 anti-syndecan-1 ectodomain antibody. In panel A, intact syndecan-1 ectodomains migrate as smears because of heterogeneous length of heparin sulfate and extent of modifications. In panel B, samples were digested with 20 mU/ml heparinase II and 20 mU/ml chodroitin sulfate ABC lyase, and then analyzed by SDS-PAGE and Western immunoblotting using the 281-2 antibody. Syndecan-1 core proteins migrate as ˜80 kDa (predicted fragment generated by constitutive shedding of syndecan-1, indicated as asterisk) and ˜60 kDa (predicted fragment generated by direct proteolysis of syndecan-1 ectodomains by exogenous proteases, indicated as arrow).

### ILLUSTRATIVE DESCRIPTION OF THE INVENTION

The present invention provides compositions and methods for detecting, treating, and preventing infection by gram negative bacteria, particularly by sporulating gram negative bacteria, especially by bacillus infection, very especially infections caused by Bacillus anthracis (“anthrax”).

The extracellular domain (ectodomain) of membrane proteins (including proteoglycans) can be released from the cell surface by a process known as ectodomain shedding. In this process, proteolysis of the membrane protein results in the cleavage of the ectodomain and its subsequent shedding or release into the extracellular environment as a soluble molecule. The present invention is related to the discovery that infection with bacillus can promote ectodomain shedding, particularly of the integral membrane proteoglycans syndecan-1 and -4. Once released into the extracellular fluid, the soluble ectodomain can contribute to pathological events associated with bacillus infection, including, e.g., inflammation, immune cell activation, and apoptosis. By modulating the amount and activity of the soluble ectodomain in an infected subject, the bacillus infection can be treated and/or prevented. As discussed in more detail below, an aspect of the present invention relates to modulation of the soluble ectodomain shed from cell surfaces in subjects infected with bacillus, particularly the syndecan ectodomain, thereby treating and/or preventing bacillus infection.

The phrase “modulating soluble ectodomain” or “modulation of soluble ectodomain” includes any process that affects the appearance and/or activity and/or quantity of soluble ectodomain in the extracellular environment. This includes, but is not limited to, e.g., directly blocking the shedding process and/or consequent shed ectodomain transformation into secondary disease mediators (e.g., using protease inhibitors that inhibit the protease from cleaving the ectodomain); blocking the signaling cascade that results in the release of soluble ectodomain (e.g., administering kinase inhibitors); neutralizing or inhibiting the activity of soluble ectodomain and products resulting from its bioconversion (e.g., using antibodies to the protein or saccharide ectodomain epitopes); inhibiting the synthesis of the membrane protein, itself (thereby reducing the amount of ectodomain available for shedding); actively removing or absorbing soluble ectodomain and products resulting from its bioconversion from the blood, blood components, or other extracellular compartments; etc.

Syndecans are of particular interest. These are cell surface heparan sulfate proteoglycans that are involved in a wide range of cellular activities, including cell binding, cell signaling, cytoskeletal organization, cell adhesion, growth factor function, and host defense. See, e.g., Bernfield et al., Ann. Rev. Biochem, 68:729-77, 1999. The basic structure comprises an extracellular ectodomain having a consensus sequence for glycosaminoglycan attachment with a protease cleavage site in the proximal region; a single hydrophobic transmembrane domain; and a C-terminus cytoplasmic domain. See, e.g., Woods and Couchman, Curr. Opin. Cell. Biol., 13:578-583, 2001. There are at least four members of this family. Syndecans-1 and -3 are present in epithelial and neuronal cells, syndecan-2 is expressed in mesenchymal cells, and syndecan-4 is expressed in a wide range of cell types. Kim et al., Mol Cell. Bio., 5, 797-805, 1994. Syndecan-1 is a transmembrane (type I) heparan sulfate proteoglycan that participates in cell proliferation, cell migration, and cell-matrix interactions via its receptor for extracellular matrix proteins.

Syndecans contains a heparan sulfate (HS) moiety attached to the ectodomain. HS is a highly anionic glucosaminoglycan heparan sulfate comprising alternating modified N-acetyl-glucosamine and glucuronic acid residues in which acetyl groups are replaced by sulfate groups. See, e.g., Gotte, FASEB J., 17:575-591, 2003. The HS chains impart a variety of functions to syndecans that involve them in morphogenesis, tissue repair, host defense, tumor development, and energy metabolism. When released into the extracellular milieu, they can be responsible for many of the deleterious effects associated with bacillus infection.

Other shed proteins associated with bacillus infection, include, but are not limited to, TGF receptors, L-selectin, CD44, IL6-receptor, transmembrane chemokines CX3CL1 and CXCL 16, TNF-alpha receptors, p75 Neurotrophil Receptor, EGF-R, heparin-binding EGF-like growth factor, and CD30.

Treating bacillus infection in accordance with the present invention can be achieved by various methods. In one embodiment, methods are provided for treating a subject infected with anthrax by administering an amount of an agent that is effective to inhibit the shedding of the syndecan ectodomain. Any agent that is capable of blocking, reducing, decreasing, etc., ectodomain shedding can be utilized. Examples, include, but are not limited to protease inhibitors, metalloproteinase inhibitors, kinase inhibitors, tyrosine kinase inhibitors, protein kinase C (PKC) inhibitors, inhibitors of ADAMs, sheddases, heparanases, etc. In this and other regards the present invention relates to all forms of anthrax infection irrespective of, in particular, the tissue initially invaded by the bacterium.

The phrase “effective amount” as used throughout this disclosure means a quantity of active agent that is useful for achieving the desired therapeutic or prophylactic effect, e.g., preventing, reducing, ameliorating, etc., any of the symptoms and/or pathological events associated with infection, such as inflammation, rash, fever, sepsis, nausea, vomiting, hemorrhagic lesions, diffuse vasculitis, tissue necrosis, death, and the like, as discussed also elsewhere herein.

Effective amounts can be determined routinely, and may vary depending upon the age, health, gender, and weight of a patient, as well as the severity, frequency, and duration of the infection. The choice of the delivery system will also guide the selection of the amounts used.

The term “treating” is used conventionally, e.g., the management or care of a subject for the purpose of combating, alleviating, reducing, relieving, improving, etc., one or more symptoms of bacillus infection. Any amount of improvement is considered useful. Treating infection also includes reducing the pathogenicity or virulence of a bacillus, since the disease symptoms are less.

In accordance with the present invention, various agents can be used to treat bacillus infection. For example, protease inhibitors which are capable of blocking or reducing the proteolytic activity of a protease that cleaves the ectodomain of a membrane protein can be utilized to treat bacillus infection. Inhibitors can block the activity of endogenous enzymes, or exogenous enzymes produced by the bacillus bacterium. The inhibitors belong to several classes depending on their activity against serine, threonine, cysteine, asparagine, or metallo proteases.

Examples of inhibitors include, but are not limited to, e.g., metalloproteinase inhibitors and hydroxamate inhibitors, tissue inhibitors of metalloproteases (TIMPs), specific neutralizing antisera and immunoglobulins, including α-macroglobulins, certain antibiotics, such as doxycycline, chelating substances, such as phenanthroline, ADAM inhibitors, kinase inhibitors, and protein kinase C inhibitors, to name but a few.

Examples of metalloproteinase inhibitors, include, but are not limited to, e.g., TIMPs, galardin, doxycycline, o-phenanthroline, phosproramidon, suramine, EDTA, EGTA, sulfonated amino acids hydroxamates, etc.

Examples of hydroxamate inhibitors include, but are not limited to, e.g., peptide hydroxamate shedddase inhibitors, BB-94 (See e.g., Holen et al., Br. J. Haematol. 2001 Aug;114(2):414-21), BB-2116, BB-1101 (British Biotechnology Co., Oxford, UK), GM6001, TAPI-1 (See e.g., U.S. Pat. No. 6,861,504), etc. See, e.g., U.S. Pat. No. 6,686,335.

Examples of ADAM inhibitors include, but are not limited to, e.g., hydroxamate GW280264X (see e.g., Budagian et al., J. Biol. Chem., 279(39):40368-75, 2004)

Examples of protein kinase C inhibitors include, but are not limited to, e.g., bisindolymaleimide I.

Examples of kinase inhibitors include, but are not limited to, e.g., tyrphostin A25 and methyl 2,5 dihydroxycinnamate (tyrosine kinase inhibitors); MAP kinase inhibitors, such as PD98059, SB202190, etc.

Antibodies and other binding moieties can also be utilized to treat infection. For example, antibodies specific for the ectodomain can be administered to infected subjects in amounts which are effective to neutralize the ectodomain activity. Ectodomain antibodies can be generated routinely, e.g., using the entire region or parts of it to elicit an immune response. The term “antibody” as used herein includes intact molecules as well as fragments thereof, such as Fab, F(ab′)2, and Fv which are capable of binding to an epitopic determinant present in the ectodomain. It also includes polyclonal, monoclonal, recombinant, chimeric, humanized, and single-chain antibodies, and fragments of any of the foregoing. These can be prepared according to any suitable method. Antibodies can be generated to the ectodomain polypeptide sequence or to antigens attached to it, e.g., sugar residues and other moieties that are attached to the polypeptide backbone.

Antibodies that can be used in accordance with the present invention (for both detection and therapeutic uses) can be raised to any epitope of an ectodomain. For instance, the primary and secondary antibodies can be raised against different epitopes of the polysaccharide portion of syndecans or (other proteoglycan ectodomain molecules), including the neo-epitopes generated in the process of proteoglycan extracellular polysaccharide degradation. In this case the extent of degradation could serve as an indicator of the disease progression.

Useful agents can act by inhibiting the activity of pathogenic factors, thereby reducing shedding. Examples of pathogenic factors include, but are not limited to, anthrax lethal toxin, anthrax hemolysins, and anthrax proteolytic enzymes. Agents which target and inhibit these factors can be used to treat anthrax infection in accordance with the present invention.

The present invention also relates to methods of identifying agents which inhibit ectodomain shedding in order to determine agents which can be used to treat bacillus infection. These methods can be applied to both in vitro and in vivo models, where bacillus infected cells are contacted with an agent, and then a reduction in ectodomain shedding is used as a marker to assess the agent's ability to treat infection.

The present invention also provides methods of treating a subject infected with bacillus, comprising: administering an effective amount of a TLR2 antagonist to the subject infected with bacillus. As discussed above, the HS component of the soluble syndecan can stimulate the toll-like receptor-2. This receptor pathway contributes to bacterial sepsis. Antagonists of TLR2 can therefore be used to treat bacillus infection. These include, e.g., T2.5 antibody (e.g., Meng et al., J. Clin. Invest., 113(10):1473-81, 2004) and other neutralizing antagonist antibodies.

While it may be believed that any of the above-mentioned agents inhibit ectodomain shedding, the present invention covers the use of the agents for treating and/or preventing bacillus infection regardless of the mechanism of action or pathway responsible for the therapeutic or prophylactic effect. Agents can be administered at any effective time before or during the course of a bacillus infection. For example, agents as mentioned above can be administered to a subject who is suspected of being infected with bacillus, but who has not shown overt symptoms. Additionally, it can be administered prophylactically to subjects who may encounter bacillus.

The enhanced, abnormal shedding of the ectodomain of syndecans into the extracellular environment can be associated with a number of pathological events. The methods of the present invention can be used to reduce, block, or decrease any one of these pathophysiological events, thereby treating and/or preventing bacillus infection.

For example, the HS moiety attached to shed soluble syndecans can activate leukocytes, and stimulate dendritic cells, leading to the inflammatory response associated with bacillus infection. Additionally, it can perturb chemokine gradients, affect leukocyte chemotaxis and migration, modulate the interaction between endothelial cells and leukocytes, and stimulate TLR signaling. For example, membrane bound syndecan can serve as a substrate to attract neutrophils and other infection fighting blood cells into the site of infection. When the syndecan is shed, the chemotactic surface is eliminated, impeding the migration of cells into the infected area. This impairs the ability of the infected host to combat the bacillus infection. Shed syndecans can also act as TLR agonists, contributing to the faulty immune response associated with bacillus infection.

The soluble ectodomain can also interfere with host defenses by inhibiting agents that mediate the innate host defense system. For example, purified syndecan ectodomains, through their HS chains, bind tightly to cationic antimicrobial peptides of the (Pro/Arg)-rich cathelicidin family and inhibit their antibacterial activities. Because several other antimicrobials in the lung are highly cationic (such as lysozyme, lactoferrin, b-defensins, LL-37) (Hunter and Bevins, 1999; Bals et al., 1999), shed ectodomains can enhance bacillus virulence by inhibiting the activity of these agents. Shed ectodomains also bind to neutrophil elastase and cathepsin G (Kainulainen et al., 1998). Elastase has been shown to be important in defending the host against gram-negative bacterial sepsis (Belaaouaj et al., 1998). Syndecan ectodomains bind to surfactant proteins A and D in a calcium-dependent manner. These surfactants belong to the collectin family of host defense molecules, and are critical in protecting the host from microbial lung infections, especially P. aeruginosa (Crouch, 1998). Finally, soluble HS can inhibit the activity of several cytokines involved in phagocyte recruitment.

In addition to treating and/or preventing bacillus infection, the present invention also provides compositions and methods for detecting bacillus infection. As discussed previously, bacillus infection initiates a pathophysiological process that results in increased ectodomain shedding. The level of shed ectodomain can be utilized as a diagnostic marker for bacillus infection. Detection methods for determining whether a subject is infected with bacillus, can comprise, e.g., detecting the presence and/or quantity of soluble syndecan in the blood of a subject suspected of being infected with bacillus, whereby the specific type of soluble syndecan indicates that the subject is infected with a particular species or strain of bacillus. The amounts of soluble ectodomain (such as syndecan) can be compared to control or standard values that establish the amount of the ectodomain in the blood (or other compartment) in normal and uninfected individuals, and/or compared to the same subject at a different stage of infection.

The amount of degradation of the cell membrane protein (and the corresponding amount of shed ectodomain) can be used to monitor the progression of the disease. As the bacillus infection advances, the quantity of a soluble ectodomain will increase in extracellular compartments (e.g., blood) and correspondingly, the amount of intact cell membrane protein from which it is shed will decrease. This progression can be followed by monitoring the appearance of shed ectodomain in the blood and/or by the appearance of neo-epitopes associated with the degradation of the membrane proteoglycan comprising the ectodomain. As discussed below, antibodies can be routinely raised against these targets and utilized in diagnostic/prognostic assays.

Soluble ectodomain can be detected, visualized, determined, quantitated, etc. according to any effective method. Useful methods include, e.g., but are not limited to, immunoassays, RIA (radioimmunoassays), ELISA, (enzyme-linked-immunosorbent assays), immunofluorescence assays, flow cytometry assays, histology assays, electron microscopy assays, light microscopy assays, immunoprecipitation assays, and Western blot assays, to name just a few.

Immunoassays may be carried out in liquid or on biological support. For instance, a sample (e.g., blood, plasma, stool, urine, cells, tissue, cerebral spinal fluid, body fluids, etc.) can be brought in contact with a solid phase support or carrier (such as nitrocellulose or plastic) that comprises an antibody or other binding agent that is capable of specifically recognizing the ectodomain of interest. The support may then be contacted with a second antibody, which also recognizes the ectodomain, preferably at a second site different from the site recognized and bound by the first antibody. The solid phase support can then be washed with a buffer a second time to remove unbound antibody. The second antibody can be detectably labeled, e.g., with a fluorescent label or an enzyme, or it can be labeled by a secondary labeling reagent that binds to it specifically, and then its presence measured by conventional means for detecting the label.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, and polyacrylamides. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc.

One of the many ways in which a polypeptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect transmembrane proteins through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the appropriate wavelength, its presence can be detected by the fluorescence of the label. Among the most commonly used fluorescent labeling compounds are fluorescein isothiocyanate, rhodamine, phycoerythrin, phycocyanin, allophycocyanin, o-phthaldehyde, and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt, and oxalate ester. Indirect as well as direct chemiluminescent methods can be used.

In addition, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that can be used for purposes of labeling are luciferin, luciferase, and aequorin.

The present invention also relates to methods of treating a subject infected with bacillus, comprising, removing soluble syndecan from the blood of a subject infected with anthrax. Numerous methods and devices have been described for the ex vivo removal of agents from various blood components by circulating blood outside of the body through an apparatus containing membranes, supports, or matrices to which are attached binding agents for the component to be removed. For example, heparinase has been attached to a particulate support to degrade heparin in blood (U.S. Pat. No. 4,373,023); chelants to remove metal ion oxidants have been described for the treatment of atherosclerosis (U.S. Pat. No. 5,753,227); and an adsorbent for removing low density lipoprotein (LDL) and endotoxins (U.S. Pat. No. 5,476,715), the endotoxin bound using a homo-, co-, or terpolymer of acrylic acid and/or methacrylic acid. See, also U.S. Pat. No. 6,365,147. Any of these methods can be utilized in the context of the present invention, e.g., where heparinase is used to remove the heparan containing soluble syndecan ectodomain.

A method for depleting syndecan from a solution, can comprise: exposing a solution to a matrix comprising a syndecan binding partner under conditions effective for syndecan in the solution to bind to the syndecan binding partner of the matrix and then separating syndecan bound to the matrix from the solution.

A further method for depleting soluble syndecan from blood, or a component thereof, can comprise: (1) providing a chromatography matrix comprising a syndecan binding partner, such as heparanase or an antibody specific for its ectodomain; (2) exposing the solution to the matrix under conditions wherein the soluble syndecan binds to the binding partner associated with the matrix; and (3) collecting the solution after exposure to the matrix, wherein the solution is depleted of syndecan. Various adsorbent materials or matrices may be used for the aforementioned purpose, in the form of beads, fibers, or other formats, comprising, by way of non-limiting example, various plastic resins such as polystyrene, polymers such as poly(hydroxymethacrylate), agarose, etc.

The present invention also provides pharmaceutical combinations for treating bacillus infection. Generally these comprise a plurality of agents which are utilized to treat a bacillus infection. Anthrax is generally treated with antibiotics, such as ciprofloxacin and derivatives thereof. Thus, any agent disclosed above can be combined with an antibiotic, and administered to infected subjects. Combinations can comprise, e.g., (a) ciprofloxacin, and (b) an effective amount of at least one agent selected from: a protease inhibitor, protein kinase C inhibitor, MAP kinase inhibitor, TLR2 receptor antagonist, and/or an antibody specific for the ectodomain. These combinations can also include therapeutic agents directed against other consequences of anthrax pathogenic factors activity, in addition to shedding, such as apoptosis, increased vascular permeability, hemorrhages, liver hypoxia, nervous system damage, renal system damage, lung edema, lymphatic system damage, impaired immune response, etc. The agents can also be administered, or co-administered with bacillus vaccines. The agents can be delivered at the same time, in a single composition, or at different times where each agent is administered alone or in combination with other active agents.

In various of its aspects and certain of the preferred embodiments thereof the invention relates to P1 and P2 proteases of Bacilluis anthracis (as discussed in the Examples below) and to proteases having any of at least 70, 75, 80, 85, 90, 95, 97, 98 or 99% identity to one or more of the terminal amino acid sequences thereof as set forth herein (see the figures, the examples and the disclosure below). In this regard, identify may be determined by any of a variety of well known and accepted software programs for determining and or calculating the degree of sequence identify of two or more amino acid or nucleic acid sequences. A preferred program for so doing, readily available to the public via the internet, is the BLAST suite of programs provided by the National Institutes of Health on the NCBI web site. In particularly preferred embodiments in this regard the parameters of the BLAST programs are set to their default values to determine the identity of the sequences. Should any ambiguities arise of a material nature regarding these programs or the parameters, for reference purposes the most preferred methods are the BLAST programs and default parameters on the NCBI BLAST programs offered for public use via the NCBI BLAST website as of the date of filing of this (PCT) application.

In various aspects and preferred embodiments the invention further relates in this regard, inter alia, to amino acid sequence variants of the foregoing, including those with conservative substitutions, non-conservative substitutions, deletions and additions, and to fragments of the aforementioned proteins and amino acid sequence variants thereof.

Further in this regard, and others, various aspects and embodiments of the invention relate to proteins formed by fusing any of the foregoing with part or all of other polypeptides to form a fusion protein, and to amino acid sequence variants and fragments thereof.

In all of the foregoing regards, among others, the invention in various aspects and preferred embodiments thereof relates to the P1 (herein referred to as Npr599) proteases having the N-terminal sequence: (1) KPVTGTNAVG or (2) VTGTNAVG, set forth in FIG. 9C and described n greater technical detail in the Examples. These sequences are the N-terminal “tags” of alternatively cleaved M4 thermolysin-like neutral protease (NP_843132), having a calculated MW of 34.1 kDa (observed MW is 36 kDa). The full length P1 gene identified by the amino acid tag (BA0599 in B. anthracis Ames genome) encodes a protein 99.3% identical to lactobacillus hydrolase (BAA06144); 99.1% identical to B. cereus neutral protease (AAZ42070), 97.7% identical to bacillolysin (YP034856), and 72.3% identical to bacillolysin MA (BAD60997), all of which belong to the neutral protease family (Npr). It also is 33% identical to Pseudomonas aeruginosa LasB (DQ150629).

In all of the immediately foregoing respects as relating to P1 proteases, the invention in various aspects and preferred embodiments thereof relates also to P2 proteases having an N-terminal sequence: (1) TGPVRGGLNG or (2) SNGTEKKSHN. In particular in this regard, the invention relates to P1 proteases having N-terminal sequence (1) that are approximately 46 kDa and those having N-terminal sequence (2) that are approximately 18 kDa.

Both of the P2 proteases (see detailed descriptions thereof in the Examples and the Figures) are members of the M6 family immune inhibitor A metalloproteases (InhA) encoded by the BA1295 gene. The 18 kDa protein (calculated MW 18.1 kDa) appears to be an autoprocessed product of an immune inhibitor A metalloprotease like that of B. cereus. This protein is designed InhA herein.

In various other aspects and preferred embodiments thereof the invention relates to any one or more of the foregoing proteases and related proteins in purified form, wherein the protease is at least 70, 75, 80, 85, 90, 95, 96, 97, 98, or 99% by weight of the purified composition, particularly referring to other proteins therein. In a particularly preferred embodiment in this regard the protease is substantially homogeneous (i.e., substantially free of other proteins). For instance, regarding the latter, in a preferred embodiment in this regard no other proteins can be detected upon SDS-PAGE followed by standard staining techniques when the sample is loaded so that the protease band(s) are just above saturation.

Further in these and other regards, the invention in various of its aspects and certain of the preferred embodiments thereof, relates to the aforementioned proteases and preparations thereof wherein the protease has an activity that is at least 25, 30, 35, 40, 45, 50, 55, 60, 65, 70, 75, 80, 85, 90, or 95% of its maximum activity and/or has a specific activity that is at least 25, 30, 35, 40, 45, 50, 55, 60, 65, 70, 75, 80, 85, 90, or 95% of its maximum specific activity.

The invention further relates in various of its aspects particularly to the use of the aforementioned proteases in assays and as targets for the development of pharmaceutical agents, such as inhibitors of their activity that can be used to decrease shedding and/or otherwise retard, ameliorate, halt and/or reverse an infection by gram negative bacteria, particular a member of Bacillus sp., especially Bacillus anthracis.

Such assay can be carried out in a very wide variety of well known methods, including those described above, many of which are highly automated and allow for the screening of large number of candidates in a relatively short period of time.

The present invention provides to methods of determining whether a subject is infected with anthrax, comprising: detecting increased levels of soluble syndecan-1 in the blood and/or tissues of a subject suspected of being infected with anthrax, whereby the presence of the increased levels of soluble syndecan-1 indicates that the subject is infected with anthrax. Methods of the above, e.g., wherein the detecting is performed by an assay, such as an immunoassay, which employs specific means of detection for epitopes of a particular soluble ectodomain or its metabolic products, such as the antibody specific for syndecan core protein.

The present invention also provides methods of treating a subject infected with anthrax, comprising: administering an amount of an agent that is effective to inhibit the shedding of the particular ectodomain, such as syndecan-1, and its further metabolism leading to the appearance of secondary mediators of toxicity. Methods of the above, wherein the agent inhibits the activity of microbial pathogenic factors causing enhanced ectodomain shedding; wherein the pathogenic factors are one or several of the following: anthrax lethal toxin, anthrax hemolysins, and/or anthrax proteolytic enzymes; wherein the agent is a protease inhibitor; wherein the protease inhibitor is a metalloproteinase inhibitor; wherein the agent is a protein kinase C inhibitor, e.g., bisindolymaleimide; wherein the agent is a MAP kinase inhibitor, e.g., PD98059, SB20219; wherein the agent is a peptide hydroxamate sheddase inhibitor, such as BB-2116, BB-1101, GM6001, or TAPI-1.

The present invention also provides methods of treating a subject infected with anthrax, comprising: removing soluble ectodomain, and/or microbial pathogenic factors causing increased ectodomain shedding, from the blood of a subject infected with anthrax, or neutralizing its activity. Methods of the above, wherein the removing is accomplished by filtering blood through a matrix comprising antibodies specific for ectodomain epitope(s).

The present invention also provides methods of treating a subject infected with anthrax, comprising: a combination therapy, which includes administration of an antibacterial substance with the substance effective in suppressing or eliminating the consequence of shed ectodomain activity.

Methods of the above mentioned methods can further include, e.g., administering along with an antibiotic, an effective amount of a protease inhibitor, protein kinase C inhibitor, MAP kinase inhibitor, or TLR2 antagonist; wherein the pathogenicity or virulence of anthrax is reduced in the subject; wherein abnormal inflammatory response leading to pathologic consequences is reduced.

The present invention also provides compositions, e.g., compositions comprising (a) ciprofloxacin, and (b) an effective amount of any of the following: a protease inhibitor, protein kinase C inhibitor, MAP kinase inhibitor, or TLR2 receptor antagonist; Substantially homogeneous Npr599; A substantially homogeneous protease comprising the N-terminal amino acid sequence KPVTGTNAVG or VTGTNAVG; Substantially homogeneous InhA; Substantially homogeneous protease comprising the N-terminal amino acid sequence TGPVRGGLNG or SNGTEKKSHN.

The present invention also provides methods for screening for a modulator of ectodomain shedding, comprising incubating a candidate inhibitor with Npr599 protease or InhA protease or both proteases and a substrate therefor and determining the effect of the candidate on substrate utilization by the protease(s).

The entire disclosure of all patents and publications, cited above are hereby incorporated by reference in their entirety.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

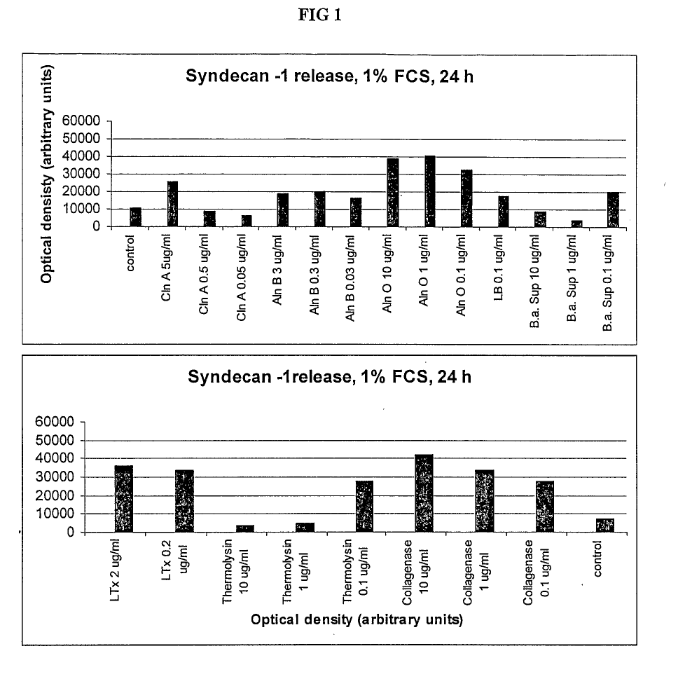

### Examples—FIGS. 1 through 8

Normal murine mammary gland epithelial cells (NMuMG, ATCC #CRL-1636) are widely accepted and used as a model for ectodomain shedding, and were used in the examples described below. In all cases, except as otherwise noted, the cells were grown to confluency in 24-well plates in media containing 1% fetal calf serum (FCS). Confluent monolayers grown as above were treated with the different B. anthracis pathogenic factors and control substances listed immediately below.

Cereolysin A (ClnA) from B. cereus is closely related to a B. anthracis enzyme AnlA, which is a phosphatidyl choline-preferring phospholipase C. It was obtained from Sigma, and used at 5, 0.5, and 0.05 μg/ml.

AnlB is a B. anthracis sphingomyelinase. It was expressed as a mature protein in E. coli cells, and isolated as a pure recombinant protein. It was used at 3, 0.3, and 0.03 μg/ml.

AnlO is a B. anthracis pore-forming hemolysin. It was expressed as a mature protein in i cells, and isolated as a pure recombinant protein. It was used at 10, 1, and 0.1 μg/ml.

LB culture media was used as a control for culture supernatant and proteins therein.

B. anthracis culture supernatant was obtained from B. anthracis (delta Ames) [pXO1—, pXO2—] cultured overnight in LB media. Cells were removed from the media by centrifugation at 8000 g. The supernatant was sterilized by filtration through a 0.22 μm cellulose acetate filtration system (Corning, N.Y.). The filtrate was concentrated 50-fold using Amicon Ultra15 centrifugal filter devices (10K cut-off pore size) (Millipore, Mass.). The proteins were used immediately after preparation or were stored at 4° C. for several days prior to use. Protein content was determined using Bradford reagent (Bio-Rad) with bovine serum albumin as standard. The supernatant was used at 10, 1, and 0.1 μg/ml.

Lethal toxin (LeTx) was reconstituted by mixing equal weight amounts of recombinant protective antigen and lethal factor (both from List Biologicals, CA). It was used at total protein concentrations of 2 and 0.2 μg/ml.

Thermolysin (EC 3.4.24.27) from Bacillus thermoproteolyticus (Sigma, MO) is partially homologous to several B. anthracis proteases, including LeTx. It was used at 10, 1, and 0.1 μg/ml.

Collagenase from Clostridium histolyticum (Clostridiopeptidase A) is partially homologous to several B. anthracis enzymes. The collagenase preparation was obtained from Sigma (MO) and also contained clostripain, a nonspecific neutral protease with tryptic activities. It was used as a positive control at 10, 1, and 0.1 μg/ml.

Following treatment of NMuMG cells as indicated above, the cells were collected and frozen at −20° C. The cells were tested for syndecan shedding by dot-blot analysis, specifically for syndecan-1 as described below. The cells also were tested for lactate dehydrogenase (LDH) release as a common measure of cytotoxicity using a detection kit sold by Roche (Roche #1644793).

The results are graphically depicted in FIG. 1. All of the tested recombinant hemolysins, along with the lethal toxin and the culture supernatant, caused a several-fold increase in shedding. The results establish that syndecan shedding is a widespread consequence of the anthrax infectious process. It is likely that this is true for other ectodomains as well.

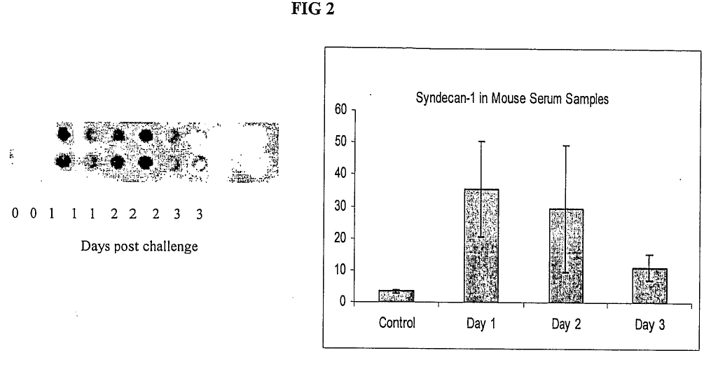

The results from analysis of the blood of B. anthracis spore-challenged mice confirm these cell culture results, as shown in FIG. 2. A strong increase in the amount of shed syndecan is detectable the day after challenge. A high level of circulating ectodomain is rapidly reached and sustained until 2 days post-infection. In the conditions of the experiment, animal death begins at day 3, judging by decrease in the signal intensity. It is accompanied by further degradation of syndecan-1 into unknown metabolites, which may be toxic.

Heparanase-mediated cleavage of syndecan-1 heparan sulfate chains could lead to reduced retention of the protein on the assay membrane, and consequently decrease the immunoblot signal. Alternatively, shed syndecan molecules may be cleared more quickly from the circulation, presumably by the liver where it also would be metabolized.

In consequence of these metabolic events, the challenged animals, expectedly, will develop something similar to a systemic inflammatory response. In particular, because degraded heparin sulfate chains are known TLR4 agonists, and activation of TLR4 typically triggers systemic immune responses. It might be expected that pathological TLR4 signaling such as this could result in liver damage in as much as the liver is highly susceptible to apoptosis.

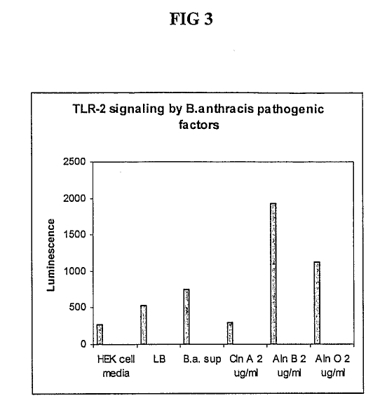

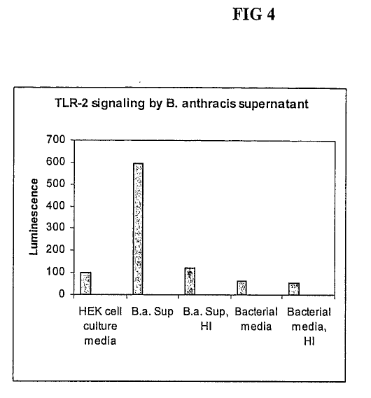

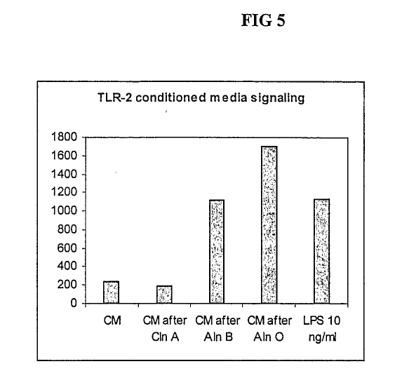

Secreted factors of B. anthracis activate TLR2 signaling in HEK 293 cells transiently transfected with the TLR2 expression construct. Upon direct treatment of transfected cells, B. anthracis culture supernatant produces a strong signal, whereas a culture media used as a control for a possible contamination with signaling substances is inactive, as shown in FIG. 3.

Whereas a heat-treated supernatant is inactive, the intensity of the signal from the test supernatants correlates with their proteolytic activity in these experiments, as shown in FIG. 4.

Signaling is abrogated when cells are stimulated in the same way in the presence of the protease inhibitor phenanthroline (previously shown by us to be a potent inhibitor of gelatinase) (data not shown).

As shown in FIG. 5, HEK cells exposed to media conditioned by NMuMG cells exposed to AnlO or AnlB acquire the capacity to signal through TLR2. The results indicate that factors expressed by NmuMG cells are shedding ectodomains from the HEK cells thereby generating TLR2 agonists, presumably syndecan-1 or heparin sulfate, but possibly other ectodomains.

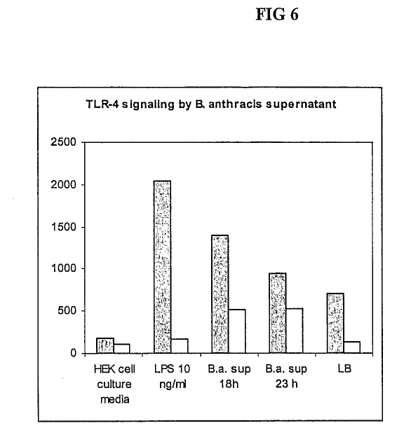

As shown in FIG. 6, shed syndecan-1 purified from the conditioned media acts as a TLR2 but not TLR4 agonist. Parallel experiments show that TLR4-transformed cells produced only a relatively weak signal when exposed to polymyxin, an inhibitor of endotoxin activity.

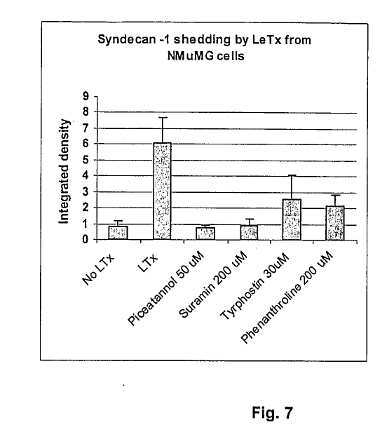

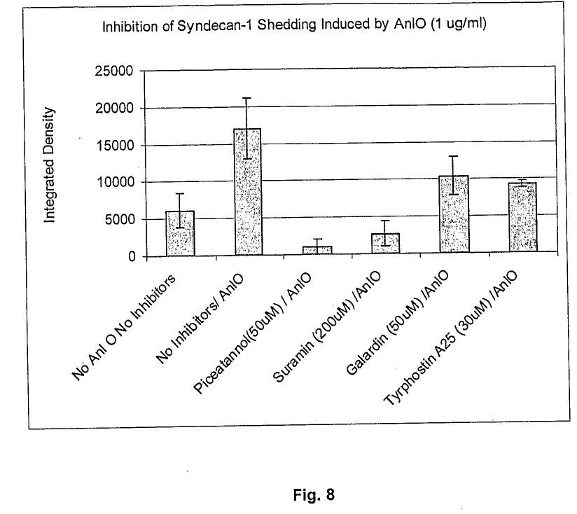

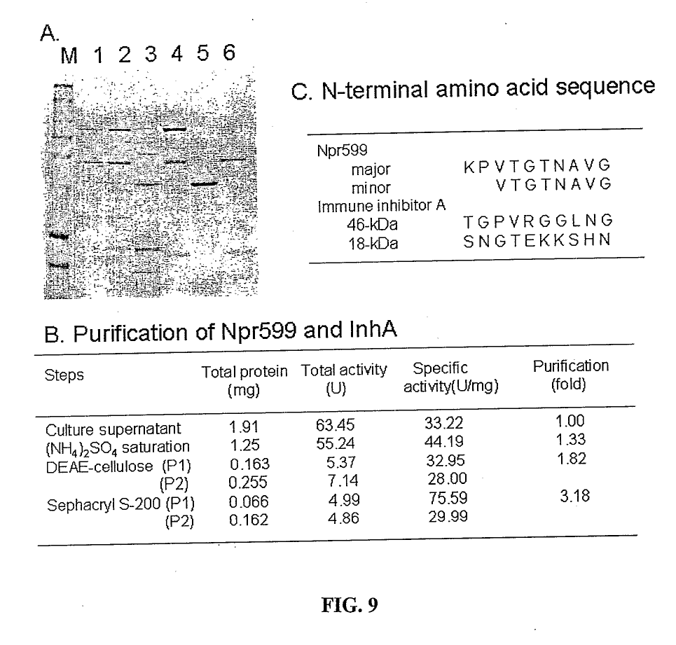

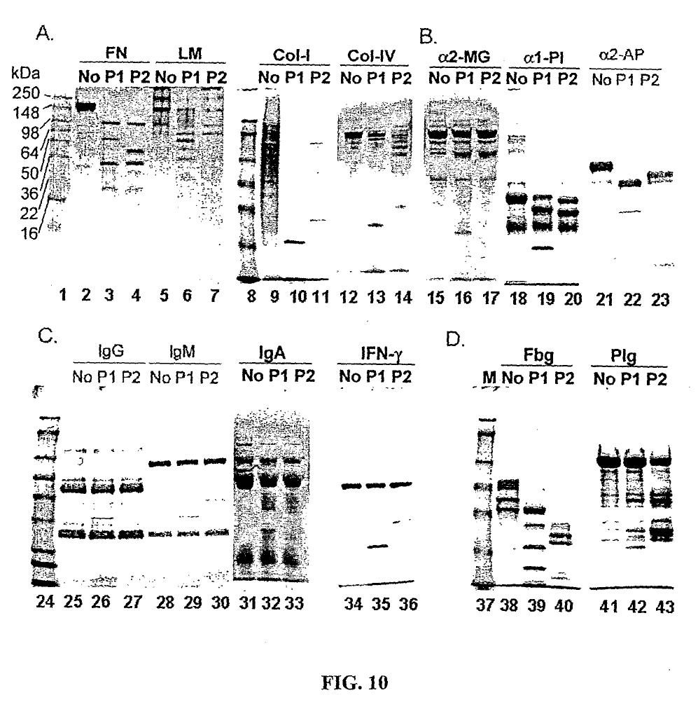

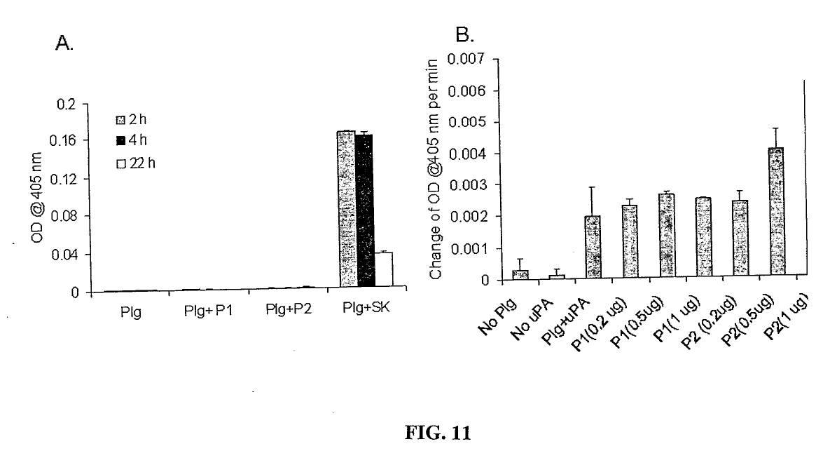

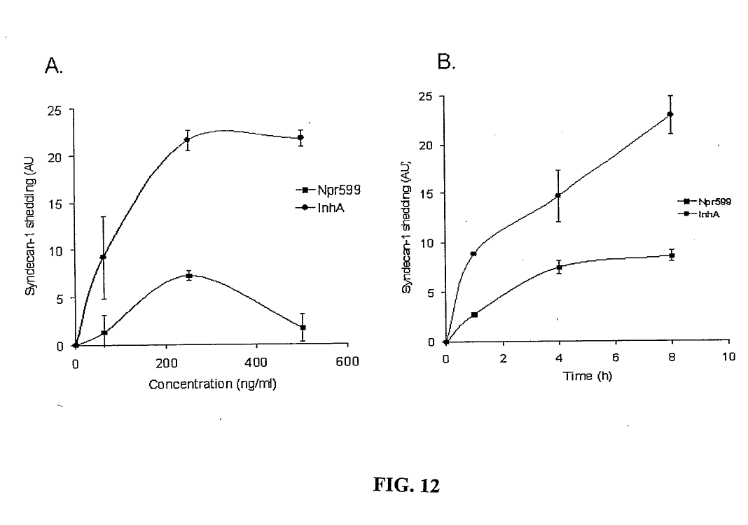

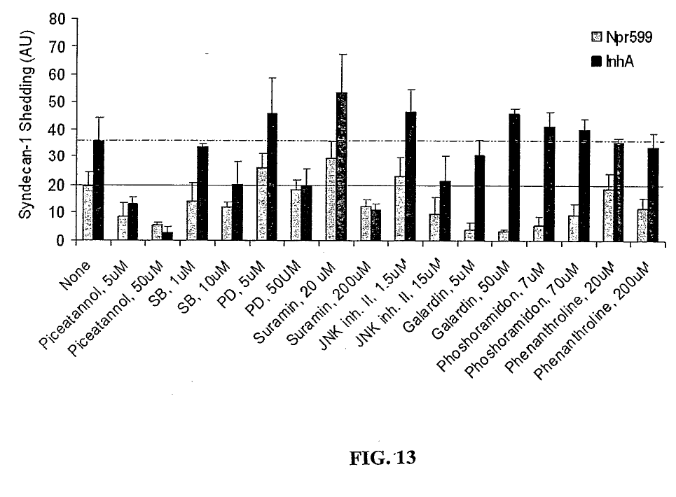

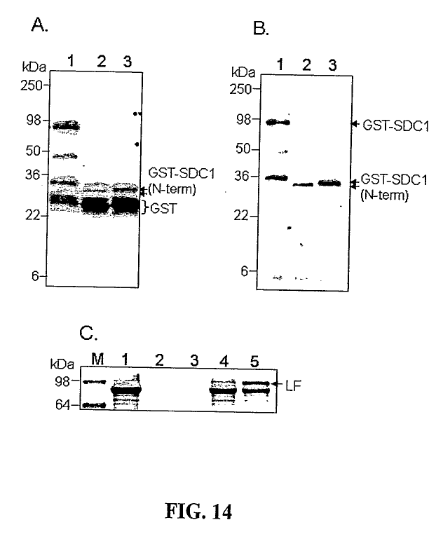

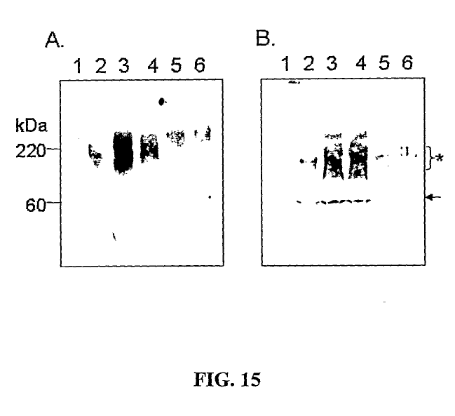

As shown in FIGS. 7 and 8, the major B. anthracis virulence factor, LeTx, and the pore-forming hemolysin, AnlO, all inhibit shedding of NMuMG cells caused by various agents. Cells were treated with the indicated amount of each inhibitor for 30 min, and then were exposed to the indicated shedding agent for 24 h in media containing 1% FCS. The amount of shed ectodomain was determined by dot blot using antibodies against syndecan-1 (281-2). The error bars indicate the standard deviations.

### Examples—FIGS. 9 through 15

The following reagents, strains, and methods were used in the following examples.

Chemicals

DEAE-cellulose (DE52), and HiPrep Sephacryl S-200 HR (26/60) gel filtration column were purchased from Whatman (Florham Park, N.J.) and Amersham Bioscience (Piscataway, N.J.), respectively. For enzyme inhibitor profile, 1, 10-phenanthroline, phenylmethanesulfonyl fluoride (PMSF), soybean trypsin inhibitor (SBTI) from Glycine max, and Galardin (Ilomastat) were obtained from Sigma (St. Louis, Mo.) phosphoramidon, pepstatin A and E-64 from Calbiochem (San Diego, Calif.), leupeptin from American Peptide Co (Sunnyvale, Calif.), and succinyl-Ala-Ala-Pro-Val-chloromethyl keton from Invitrogen-Molecular Probes (Carlsbad, Calif.). The fluorescently labeled casein, gelatin, and elastin were from Invitrogen Molecular Probes (Carlsbad, Calif.). Protein substrates calf skin type I collagen, bovine fibronectin, bovine laminin, human immunoglobulin (Ig) G, human IgM, human IgA, human plasminogen, human α₁-protease inhibitor, α₂-antiplasmin, and human fibrinogen were from Sigma, human α₂-macroglobulin from Serva (Heidelberg, Germany), recombinant human interferon-γ from R&D Systems (Minneapolis, Minn.), and human type IV collagen from Calbiochem, respectively. Val-Leu-Lys-p-nitroanilide (pNA), a synthetic plasmin substrate, was from Sigma. Recombinant streptokinase was from EMD Biosciences (San Diego, Calif.). Precast 10% and 14% SDS-PAGE gel was from Invitrogen-Novex (Carlsbad, Calif.). Plasmid for recombinant rat syndecan-1 with a GST-tag at the N-terminus was cordially provided by Dr. E. S. Oh (Ewha Women's University, Korea). Recombinant syndecan-1 protein was prepared from a host E. coli BL21 (DE3) cells through a glutathione-sepharose column.

Microbial Strain, Cultivation, and Supernatant Preparation

The non-encapsulated, atoxigenic Bacillus anthracis strain (delta Ames, pXO1⁻, pXO2⁻) was streaked on solid LB medium and isolated a single colony, followed by inoculating in a liquid LB media to obtain a seed culture. The overnight seed culture (50 ml) was inoculated and cultured in 1 L of LB at 37° C. with vigorous agitation until the cells had reached stationary phase. The culture broth was centrifuged at 17,000 g for 10 min, and the resulting supernatant was further clarified through a 0.22 μm cellulose acetate filtration system.

Protease Assays

Protease activity was measured using EnzChek Ultra Protease kits for casein hydrolytic activity, EnzChek Gelatinase/Collagenase kits for gelatin hydrolytic activity, and EnzChek Elastase kits for elastin hydrolytic activity, respectively, according to the manufacturer's recommendation. Briefly, 5 μl of supernatant or fractions in 45 μl of digestion buffer were mixed with 50 ml of fluorescein-labeled substrate, then fluorescence intensity was measured after 1 hour incubation at 37° C. using 485 nm excitation and 510 nm emission wavelengths. One unit of protease activity was defined as the amount of protease required to liberate 1 mmole of the fluorescent dye from substrate-dye conjugates in 1 min.

SDS-PAGE and Determination of Protein Concentration

Proteins were separated by SDS-PAGE in precast 14% or 10% gels under reducing and denaturing conditions according to the manufacturer's instructions. The gels were stained using Coomassie brilliant blue R-250 and then destained. Protein concentration was colorimetrically determined by the Bradford method using BioRad Protein Assay dye reagent from a standard curve of bovine serum albumin.

Characterization of the Proteases

Temperature and pH optima. To study the effect of pH on the protease activity, the proteases were assayed at 37° C. in buffers with various pH ranges containing 0.1 M NaCl; 50 mM sodium acetate-acetic acid buffer for pH 4-5.5, MES-NaOH buffer for pH 6-7, and 50 mM Tris-HCl for pH 7.5-10. Optimal temperature was determined by measuring caseinolytic activity of the protease at 21, 37, 50, and 70° C. for 1 h. For testing the effects of inhibitors on the protease activity, the proteins were pre-incubated with inhibitors, divalent ions, or other chemicals in 10 mM Tris-HCl, pH 7.8 for 30 min at room temperature. Then, an equal volume of 2× casein substrate was added, followed by further incubation at 37° C. for 1 h.

N-Terminal Amino Acid Sequencing

Partial N-terminal amino acid sequencing of the purified proteases was performed on polyvinylidene difluoride-electroblotted proteins at Midwest Analytical Inc. (St. Louis, Mo.) using an automated Edman degradation sequencer from Applied Biosystems (Foster City, Calif.).

Protease Substrate Analysis

Approximately 0.2 μg of proteases was incubated for 4 hours at 37° C. with various proteins including recombinant syndecan-1 in 20 mM Tris-HCl (pH 7.4) containing 1 mM CaCl₂ and 1 mM MgSO₄. Substrate only controls were included in parallel. Digested substrates were separated by 14% or 10% SDS-PAGE.

Plasminogen Activation

Plasminogen activation in the presence of plasma fibrin was assayed by determining Val-Leu-Lys-pNA hydrolysis. Human plasminogen (8.3 μg) was incubated at 37° C. with 2 μg of the protease or streptokinase (positive control) in 50 μl of 50 mM Tris-HCl, pH 7.5, containing 1 mM CaCl₂. The resulting reactions were diluted 20 fold and added to 100 μM Val-Leu-Lys-pNA (50 μl) in the presence of fibrin. Urokinase-type plasminogen activator (uPA)-catalyzed plasminogen activation was achieved by adding 200 U/ml uPA, 0.1 U/ml plasminogen, 100 μM Val-Leu-Lys-pNA with 2, 5, or 10 μg/ml of the purified proteases to the reaction solutions (100 μl). The release ofpNA from the chromogenic substrate was monitored at 405 nm.

Shedding Assays in Cultured Cells

Quantification of syndecan-1 shedding from NMuMG cells was performed as described previously. Briefly, cells were grown up in Dulbecco's modified Eagle's medium in 96-well plates, cultured to 1 day post confluence, then stimulated with indicated proteins using serum-free media. After stimulation, culture supernatants (100 μl) were collected and acidified with 1 ml of acidification buffer (150 mM NaCl, 50 mM NaOAc, 0.1% Tween-20, pH4.5). Cell viability was measured by lactate dehydrogenase (LDH) release using a cytotoxicity detection kit (Roche, Germany) according to manufacturer's recommendation. Samples were applied to Immobilon NY+ membrane using a Bio-Dot microfiltration apparatus (Bio-Rad, CA). Washing with acidification buffer, the membrane was then incubated with rat anti-mouse syndecan-1 antibody followed by incubating with goat anti-rat HRP-conjugated secondary antibody. The membranes were developed using ECL Plus Western Blotting Detection kit (Amersham Biosciences, NJ) and Kodak BioMax Light Film (Sigma, MO). The results were quantified by scanning the exposed film, and evaluating the intensity of exposed dots by software AlphaEase FC (Alpha Innotech, San Leandro, Calif.). Results were expressed as the amount of syndecan-1 shed in relative absorbance units (AU) using a calibration curve generated by two-fold dilutions of culture supernatants from mouse epithelial cells treated with anthrolysin O. The AUs varied between different experiments because of the exposition conditions and other treatment parameters. Each AU measurement represents the mean and the 95% confidence intervals calculated using the Student t-test.

Western Blot of Syndecan-1 Eectodomains

Conditioned media from stimulated NMuMG cells for 4 h with purified proteases (250 ng/ml) or PMA (1 μM) were collected, and 1.3% (w/v) potassium acetate and 3 volume of 95% EtOH were added to the media. After overnight at −20° C., samples were dissolved in digestion buffer (100 mM Tris-HCl, pH 8.0, 0.1% Triton X-100, 5 mM EDTA, and 1 mM PMSF) and half volume of each samples were digested with 20 mU/ml heparinase II and 20 mU/ml chondroitin sulfate ABC lyase at 37° C. overnight. The digested and undigested samples were fractionated by SDS-PAGE using 4-20% gradient acrylamide gels, electrophoretically transferred to Immobilon NY+ nylon membrane. Membranes were probed with monoclonal rat anti-mouse syndecan-1 antibodies (281-2), and then HRP-conjugated goat anti-rat IgGs, and developed by the ECL detection method.

Results depicted in FIGS. 9 through 15 were obtained as described below.

Purification and Characterization of Npr599 and InhA

Secreted proteases were purified from B. anthracis as follows. The non-encapsulated, atoxigenic Bacillus anthracis strain (delta Ames, pXO1⁻, pXO2⁻) was streaked on solid LB medium and isolated as a single colony. The colony was inoculated in a liquid LB media to obtain a seed culture. The seed culture was expanded and then cultured in nutrient-limiting medium Luria Broth (LB) at 37° C. with vigorous agitation until the cells reached stationary phase. The cells were collected by centrifugation at 17,000 g for 10 min. The culture supernatant was clarified by passing it through a 0.22 μm cellulose acetate filtration system.

All subsequent operations during the enzyme purification were performed at 4° C. unless otherwise indicated. Solid ammonium sulfate was added to the culture supernatant to 75% saturation. Precipitated proteins were collected by centrifugation at 17,000 g for 20 min. The precipitate was dissolved in 50 mM Tris-HCl (pH 7.6) containing 3 mM sodium azide, and then dialyzed against the same solution. The dialyzate was loaded onto a DEAE-cellulose anion exchange column (bed volume=60 ml) equilibrated with 50 mM Tris-HCl (pH 7.6) containing 3 mM sodium azide. Step wise fractions were eluted with buffer containing 10, 50, 100, 200, 500, and 1,000 mM NaCl. Two protease fractions were obtained, with activities against casein and elastin: P1, the flow through fraction, and P2, the 200 mm NaCl eluate. Both fractions were purified to apparent homogeneity by HPLC on a Sephacryl S-200 gel filtration column equilibrated with 20 mM Tris-HCl (pH7.6) containing 150 mM NaCl, run at a flow rate of 1.3 ml/min and collecting 5 ml fractions. The fractions were assayed for protease activity as described above and further characterized as described below.

In the reduced denaturing SDS-PAGE, the purified enzymes show a single protein band for P1 with a molecular mass of 36 kDa, and two protein bands for P2 with molecular masses of 46 and 18 kDa, which were copurified on the chromatography (FIG. 9A). Overall purification of the proteases from the culture supernatant of B. anthracis is summarized in FIG. 9B. The proteases are highly abundant, and therefore their purification rate over the crude culture supernatant is 3.2.

To identify the proteases and to determine if the isolated proteins correspond to the particular maturation forms of preproenzymes, we sequenced the N-terminal amino acids by an automated Edman degradation. It was determined that P1 protease contains KPVTGTNAVG as a major sequence and VTGTNAVG as a subsequence (FIG. 9C). It identifies theses sequences as the alternatively cleaved N-terminal parts of the catalytic domain of the M4 thermolysin-like neutral protease (NP_{—}843132), with the calculated MW of 34.1 kDa (observed MW is 36 kDa). The full P1 gene (BA0599 in B. anthracis Ames genome) encodes the protein, which is 99.3% identical to lactobacillus hydrolase (BAA06144), B. cereus neutral protease (AAZ42070, 99.1% identity), bacillolysin (YP034856, 97.7% identity) and bacillolysin MA (BAD60997, 72.3% identity), all of which belong to the neutral protease family (Npr). It has low homology (33%) with Pseudomonas aeruginosa LasB (DQ150629). The amino acid sequences at putative signal peptide cleavage sites, propeptide cleavage sites, zinc binding sites, and active sites in P1 and the above Npr proteins are highly homologous. We designated P1 as Npr599 herein.

The N-terminal sequences of isolated P2 protease were determined to be TGPVRGGLNG for the 46 kDa protein and SNGTEKKSHN for the 18 kDa protein (FIG. 9C). Both of the proteins belong to the M6 family immune inhibitor A metalloproteases (InhA) encoded by the BA1295 gene. The 18 kDa protein (calculated MW 18.1 kDa) appears to be an autoprocessed product of an immune inhibitor A metalloprotease like that of B. cereus. We designated this protein as InhA.

Zn-Metalloprotease Activities of Npr599 and InhA

The caseinolytic activities of Npr599 and InhA were assayed in the range of buffers with pH from 4 to 10. The highest activity at 37° C. was found in the Tris-HCl buffer in the interval of pH from 7 to 8, indicating that the isolated enzymes belong to the class of neutral proteases.

To estimate the optimal temperature, the proteases were assayed for caseinolytic activity at 21, 37, 50, and 70° C. at pH 7.8 in Tris-HCl buffer (pH 7.8) without adjusting pH for each temperature. Both of the enzymes display high activity at 37° C., and remain fully active at 50° C.

The effect of various inhibitors on activity of these proteases is enumerated in Table 1. Both Npr599 and InhA are rapidly inhibited by metal-chelating agents such as EDTA and 1,10-phenanthroline. InhA is less sensitive to phosphoramidon and galardin, compared to Npr599. DTT, a strong disulfide bond reducing agent, inhibits both proteases, but milder thiol reducing compounds like β-mercaptoethanol and L-cystein (at 1 mM) show no substantial inhibiting activity. These results suggests the presence of disulfide bonds important to conformation.

3.5 μM SDS activates Npr599 approximately 2.4-fold, similar to the effect of Brij 35 on the leukocyte elastase activity. The effect of these detergents may mimic a biologically-relevant activation mechanism. The divalent metal ions Cu²⁺, Fe²⁺ and Zn²⁺ inhibit the caseinolytic activities of Npr599 and InhA, whereas Ca²⁺, Mg²⁺ and Mn²⁺ do not (See Table 2). Nonetheless, both enzymes require zinc for hydrolytic activity: 1 mM 1,10-phenanthroline depletion of the metal ion from the active center completely abolishes the activity against casein, and it cannot be restored by addition of excess (1 mM) CaCl₂ Furthermore, both Npr599 and InhA contain a HEXXH motif, which is defined as a Zn-binding domain of metalloproteases. In sum, the activity data and the primary structure-based identification both indicate that Npr599 and InhA are M4 and M6 Zn-metalloenzymes, respectively.

Npr599 and InhA Protease Substrates

To evaluate possibility of the proteases as pathogenic factors, we next surveyed their target molecules that are related to inflammation and innate immune response. When the internally quenched fluorescent substrates of casein, gelatin and elastin were used as substrates, Npr599 has strong activity for casein (14.09 U/mg) and elastin (17.48 U/mg) and relatively weak activity for gelatin (6.47 U/mg), while InhA has strong activity for casein (14.26 U/mg) and gelatin (16.28 U/mg) but relatively weak activity for elastin (4.25 U/mg). Since bacterial protease may cause tissue damage by directly degrading host tissues, significant host proteins were tested as substrates of the purified proteases. For example, the extracellular matrix proteins such as fibronectin, laminin, type I and IV collagens, which could be degraded during inflammation and bacterial infections, are candidate targets of B. anthracis proteases. FIG. 10 shows that indeed both Npr599 and InhA effectively cleave fibronectin and type I collagen, while Npr599 is more active with laminin, and less active with collagen type IV, compared to InhA. In addition to the extracellular structural proteins, α₂-macroglobulin, α₂-antiplasmin and α₁-protease inhibitor are the most important serum protease inhibitors regulating the activity of plasmin and blood elastase. FIG. 10 shows that both of these proteins are partially degraded by the proteases, which could potentially have high pathological relevance. On the other hand, the purified proteases did not prominently digest immunoglobulin A (IgA), IgG, IgM, and interferon-γ in which are important components of mucosal and T cell immunity (FIG. 10). With regard to the blood coagulation cascade, fibrinogen chains of Aα- and Bβ-type are completely cleaved by Npr599 within 4 h, unlike the γ-chains, which remain visible in the gel. On the other hand, all fibrinogen chains Aα-, Bβ- and γ-chains are completely cleaved by InhA.

TABLE 1
Effect of Protease Inhibitors on Npr599 and InhA Activity

| Inhibitors and | Conc. | Remaining protease activity (%) | | | | |
|---|---|---|---|---|---|---|
| chemicals | mM | Npr599 | | InhA | | Use recommended |
| Control | | 100 | | 100 | | |
| EDTA | 10 | 6.5 | (0.3) | 28.2 | (5.0) | Metallo |
| | 1.0 | 9.5 | (0.8) | 28.7 | (1.4) | |
| | 0.1 | 10.5 | (1.1) | 33.1 | (8.0) | |
| 1,10-phenanthroline | 10 | 0 | (0.0) | 0 | (0.0) | Metallo |
| | 1.0 | 3.3 | (0.0) | 0 | (0.0) | |
| | 1 | 55.3 | (4.8) | 34.3 | (13.8) | |
| Phosphoramidon | 5.0 | 1.7 | (0.2) | 45.3 | (1.5) | Metallo |
| | 0.5 | 2.4 | (0.5) | 80.3 | (0.8) | |
| | .05 | 12.3 | (0.0) | 87.1 | (1.6) | |
| Galardin | 1.28 | 0 | (0) | 70.5 | (1.5) | Metallo |
| | 0.128 | 12 | (0.8) | 104 | (1.3) | |
| | 0.0128 | 37.1 | (0.8) | 103.3 | (3.2) | |
| PMSF | 10 | 88.2 | (10.2) | 111.1 | (10.7) | Serine |
| | 1.0 | 100.9 | (5.4) | 109.8 | (8.5) | |
| | 0.1 | 87.2 | (2.3) | 94.7 | (2.2) | |
| Leupeptin | 10 | 95.4 | (5.1) | 75.1 | (5.9) | Serine |
| | 1.0 | 106.2 | (3.2) | 105.6 | (2.8) | |
| | 0.1 | 106.2 | (2.6) | 111.4 | (3.5) | |
| Pepstatin A | 5.0 | 67.6 | (2.2) | 31.6 | (0.9) | Acid (carboxylic) |
| | 0.5 | 75.3 | (2.9) | 85.9 | (3.1) | |
| | .05 | 77.9 | (0.5) | 88.4 | (4.7) | |
| E-64 | 5.0 | 74.1 | (0.5) | 59.1 | (1.8) | Thiol (cysteine) |
| | 0.5 | 76.1 | (0.1) | 78.4 | (7.5) | |
| | .05 | 79.5 | (3.9) | 83.5 | (0.4) | |
| SDS | 0.35 | 21.3 | (0.6) | 15.3 | (1.8) | Surfactant |
| | 0.035 | 86.5 | (17.2) | 21.6 | (7.5) | |
| | 0.0035 | 243.2 | (5.8) | 131.9 | (0.4) | |
| L-Cysteine.HCl | 10 | 0 | (0.0) | 0 | (0.0) | Cysteine |
| | 1.0 | 108.2 | (6.8) | 100 | (2.7) | |
| | 0.1 | 118.9 | (4.1) | 116 | (1.1) | |
| b-mercaptoethanol | 10 | 85.4 | (11.8) | 85.8 | (13.3) | Sulfohydryl |
| | 1.0 | 133.4 | (7.6) | 105.8 | (8.0) | |
| | 0.1 | 120.7 | (6.6) | 113.4 | (9.4) | |
| DTT | 10 | 35.2 | (2.7) | 14.8 | (3.5) | Sulfohydryl |
| | 1.0 | 83.8 | (2.8) | 65 | (8.7) | |
| | .10 | 123.2 | (1.2) | 114.8 | (11.3) | |

TABLE 2
Effect of Divalent Ions on Npr599 and InhA Activity

Remaining activity (%)

| Divalent ion | Concentration (mM) | Npr599 | InhA |
|---|---|---|---|
| Control | | 100 | 100 |
| Ca2+ | 1 | 86 (1.5) | 91 (5.2) |
| | 0.1 | 97 (1.4) | 94 (3.5) |
| Cu2+ | 1 | 0 (0.0) | 0 (0.0) |
| | 0.1 | 1 (1.0) | 0 (0.0) |
| Fe2+ | 1 | 0 (0.3) | 0 (0.0) |
| | 0.1 | 63 (1.8) | 48 (5.2) |
| Mg2+ | 1 | 83 (2.8) | 76 (6.1) |
| | 0.1 | 97 (1.4) | 97 (1.7) |
| Mn2+ | 1 | 79 (0.2) | 103 (2.5) |
| | 0.1 | 96 (4.3) | 91 (5.1) |
| Ni2+ | 1 | 46 (1.4) | 45 (3.4) |
| | 0.1 | 77 (1.4) | 69 (1.3) |
| Zn2+ | 1 | 21 (0.7) | 0 (0.0) |
| | 0.1 | 78 (2.3) | 51 (6.2) |

InhA Modulates Plasmin Activity and Blood Coagulation

As mentioned above, bacterial proteases can activate mammalian plasminogen system to induce fibrinolysis and ECM degradation. We next investigated if protease-mediated cleavage of plasminogen generates plasmin activity. As shown in FIG. 10, InhA is more active than Npr599 in cleaving human plasminogen, and produces a cleavage pattern of 5 major bands similar to that of bacillolysin MA. Then, we analyzed protease-catalyzed plasmin activity using a chromogenic synthetic substrate Val-Leu-Lys-p-nitroanilide. The degradation of plasminogen does not activate the plasmin activity, in contrast to streptokinase of Staphylococcus aureus used as a positive control (FIG. 11A). This demonstrates that Npr599 and InhA itself are not a bacterial plasminogen activator. On the other hand, in the incubation of plasminogen with urokinase-plasminogen activator (u-PA), the addition of InhA elevated the initial rate of u-PA-mediated plasminogen activation (FIG. 11B). This result suggests that InhA, but not Npr599, is a modulator of u-PA-catalyzed plasminogen activation. Along with direct cleavage of endogenous plasmin inhibitors α₂-macroglobulin and α₂-antiplasmin as shown in FIG. 10, InhA may act as a modulator of plasmin activity during anthrax infection.

Taken together, these data demonstrate that direct proteolytic effects of InhA during the infectious process are likely to prevent initiation of both blood coagulation and clot fibrinolysis through a modulation of the host's plasmin-mediated inflammation system.

Npr599 and InhA Both Activate Host Cell Syndecan-1 Shedding Activity

Proteolytic activity of Npr599 and InhA against components of extracellular matrix prompted us to evaluate effects of these proteases on intercellular interactions in epithelial monolayers. We were specifically interested in the fate of syndecan-1 ectodomains, which are involved in the maintenance of barrier permeability, cytoskeleton organization, intercellular signaling, and have been recently implicated as mediators of lethality perturbing different mechanisms of the host defense response. We tested whether anthrax extracellular proteases can modulate syndecan-1 shedding from host cells using a culture of normal murine mammary gland (NMuMG) epithelial cells. FIG. 12 shows that both Npr599 and InhA can function as sheddases releasing soluble syndecan-1 molecules into culture media in a time- and dose-dependent manner. Maximum stimulation is reached at a concentration of 250 ng/ml for both Npr599 (˜7-fold increase) and InhA (˜22-fold increase) (FIG. 12A). Furthermore, shedding activation by Npr599 is rapid and saturable by 8 hrs, whereas InhA is not saturable by the time point (FIG. 12B). At high concentrations (>250 ng/ml), syndecan-1 shedding by Npr599 is rather decreased in dot-blot analysis (FIG. 12A). Both Npr599 and InhA are shown to have minimal toxic effects on host cells when tested using LDH release assay (data not shown).

Since ectodomain shedding by host cells are inhibited by a variety of substances active in a number of receptor- and stress-activated signaling pathways, which involve protein tyrosine kinases (PTKs), protein kinase C (PKC), and mitogen-activated protein kinases (MAPKs), we next analyzed shedding activity after administering with those inhibitors in order to elucidate shedding mechanism. Shedding by both Npr599 and InhA was strongly inhibited by piceatannol, a specific inhibitor of the Syk family PTKs (FIG. 13), indicating that PTK activity is essential for the proteases-activated shedding. In order to understand which signaling pathways among p38, ERK and JNK are involved in protease-mediated acceleration of Synd shedding, we tested SB202190, an inhibitor of p38; PD98059, an inhibitor of MEK1/2 (ERK pathway); and the JNK inhibitor II. As shown in FIG. 13, low concentration of PD98059 and JNK inhibitor (5 μM) shows some stimulatory effect on syndecan-1 shedding, but it strongly inhibits syndecan-1 release in concentrations typical for its activity range of 5 to 50 μM. The inhibition experiments demonstrate that Npr599 and InhA of B. anthracis induce syndecan-1 shedding through activation of cytoplasmic PTKs followed by influencing MAPK pathways.

Suramin is an antitumoral agent that blocks the growth factors binding to several receptors, including the ones for epidermal growth factor (EGF), platelet derived growth factor (PGDF), insulin growth factor II, and transforming growth factor-b (TGF-β). These growth factors bind to heparan sulfate-containing proteoglycans (HSPGs), which can be shed in various pathophysiological processes, such as wound repair, and microbial infections. FIG. 13 shows that similar to piceatannol, suramin stimulates syndecan shedding at 20 μM. At higher concentration, suramin effectively inhibits syndecan-1 shedding induced by proteases, suggesting that Npr599 and InhA inhibit binding of growth factor binding to HSPG of cell surface receptors.

On the other hand, metalloprotease (sheddase) inhibitors galardin, phenanthroline and phosphoramidon abrogate Npr599-activated, but not InhA-activated, syndecan-1 shedding (FIG. 13). Of note, peptide hydroxamate sheddase inhibitor galardin significantly inhibits syndecan-1 ectodomain shedding triggered by Npr599, but not by InhA. This effect is consistent with differential inhibitory activity of galardin for Npr599 and InhA; galardin inhibits Npr599 activity strongly, but not InhA activity significantly as shown in Table 1. This suggests that in addition to the host cell's shedding mechanism, there is the other shedding mechanism involved in cleavage of syndecan-1 ectodomain such as direct proteolytic cleavage by exogenous proteases.

Npr599 and InhA Directly Accelerate Syndecan-1 Shedding

To investigate whether Npr599 and InhA directly cleave the ectodomain of syndecan-1, we prepared recombinant rat syndecan-1 tagged with glutathion S-transferase (GST) at the amino terminal and expressed in E. coli BL21 host cells. The GST-syndecan-1 was purified through glutathione-sepharose 4B beads. When incubated with Npr599 and InhA, GST-syndecan-1 protein is completely degraded within an hour (FIG. 14A). However, lethal factor, a metalloprotease component of lethal toxin, has no significant activity on syndecan-1 proteolysis (FIG. 14C). To identify the degraded fragments, Western blot analysis is performed using anti-GST and anti-syndecan-1 antibody (N-18) raised against a 15-20 amino acid peptide which maps within the first 50 amino acids of syndecan-1 of mouse origin. As shown in FIG. 14B, the major digestion product is approximately a 32-kDa fragment with little different sizes generated by Npr599 and InhA. This suggests that recombinant GST-syndecan-1 protein is cleaved at the site adjacent to the amino terminus, right after heparan sulfate attachment sites. To corroborate this, analysis of in vivo shed syndecan-1 fragments is supposed to be carried out by comparison with MW of shed syndecan-1 using Npr599 and InhA treated medium of cells. We therefore analyzed the size of the syndecan-1 ectodomains shed by the purified proteases and B. anthracis culture supernatants in either LB or LB containing 0.5% glucose. Supernatants from LB cultures have the highest proteolytic activity, while supernatants from LB cultures in the presence of 0.5% glucose have no proteolytic activity at all (data not shown). Therefore, culture supernatants in LB containing 0.5% glucose were used as a negative control for proteolytic activity of culture supernatants. FIG. 15 shows that both intact ectodomains (panel A) and heparinase II- and chondroitinase ABC-digested core proteins (panel B) shed by both purified proteases and culture supernatants from LB are different in size to that of the constitutively shed ectodomains (A, lane 1). However, ectodomains shed by PMA or protease-null culture supernatants from LB containing 0.5% are similar in size, which is activated by a shedding mechanism that is similar to that used for the endogenous shedding of syndecan-1 ectodomains. Of note, treatment with purified proteases or protease-positive culture supernatants generates a small MW of fragment to the conditioned media. Together with N-terminal proteolysis of recombinant syndecan-1, these findings suggest that Npr599 and InhA can further accelerate syndecan-1 shedding through direct proteolytic cleavage of ectodomain.

What is claimed is:

1. A method of determining whether a subject is infected with anthrax, comprising:
detecting increased levels of soluble syndecan-1 in the blood and/or tissues of a subject suspected of being infected with anthrax, whereby the presence of the increased levels of soluble syndecan-1 indicates that the subject is infected with anthrax.

2. A method of claim 1, wherein the detecting is performed by an assay, such as an immunoassay, which employs specific means of detection for epitopes of a particular soluble ectodomain or its metabolic products, such as the antibody specific for syndecan core protein.

3. A method of treating a subject infected with anthrax, comprising:
administering an amount of an agent that is effective to inhibit the shedding of the particular ectodomain, such as syndecan-1, and its further metabolism leading to the appearance of secondary mediators of toxicity.

4. A method of claim 3, wherein the agent inhibits the activity of microbial pathogenic factors causing enhanced ectodomain shedding.

5. A method of claim 3, wherein the pathogenic factors are one or several of the following: anthrax lethal toxin, anthrax hemolysins, and/or anthrax proteolytic enzymes.

6. A method of claim 3, wherein the agent is a protease inhibitor.

7. A method of claim 3, wherein the protease inhibitor is a metalloproteinase inhibitor.

8. A method of claim 3, wherein the agent is a protein kinase C inhibitor.

9. A method of claim 3, wherein the agent is a MAP kinase inhibitor.

10. A method of claim 3, wherein the agent is a peptide hydroxamate sheddase inhibitor.

11. A method of treating a subject infected with anthrax, comprising:
removing soluble ectodomain, and/or microbial pathogenic factors causing increased ectodomain shedding, from the blood of a subject infected with anthrax, or neutralizing its activity.

12. A method of claim 11, wherein the removing is accomplished by filtering blood through a matrix comprising antibodies specific for ectodomain epitope(s).

13. A method of treating a subject infected with anthrax, comprising:
a combination therapy, which includes administration of an antibacterial substance with the substance effective in suppressing or eliminating the consequence of shed ectodomain activity.

14. A method of claim 13, comprising:
administering along with an antibiotic, an effective amount of a protease inhibitor, protein kinase C inhibitor, MAP kinase inhibitor, or TLR2 antagonist.

15. A method of any of claims 4-15, wherein the pathogenicity or virulence of anthrax is reduced in the subject.

16. A method of any of claims 4-15 wherein abnormal inflammatory response leading to pathologic consequences is reduced.

17. A pharmaceutical combination comprising: (a) ciprofloxacin, and (b) an effective amount of any of the following: a protease inhibitor, protein kinase C inhibitor, MAP kinase inhibitor, or TLR2 receptor antagonist.

18. Substantially homogeneous Npr599.

19. A substantially homogeneous protease comprising the N-terminal amino acid sequence KPVTGTNAVG or VTGTNAVG.

20. Substantially homogeneous InhA.

21. A substantially homogeneous protease comprising the N-terminal amino acid sequence TGPVRGGLNG or SNGTEKKSHN.

22. A method for screening for a modulator of ectodomain shedding, comprising incubating a candidate inhibitor with Npr599 protease or InhA protease or both proteases and a substrate therefor and determining the effect of the candidate on substrate utilization by the protease(s).

23. A modulator of Npr599 and/or InhA protease identified by a method according to claim 22.

24. A treatment for an infection caused by a gram negative bacterium, comprising administering to a subject suffering from an infection by a gram negative bacterium by an effective route an amount of a modulator identified by a method according to claim 23 effective to treat the infection.
